(19) United States
(12) Patent Application Publication
(10) Pub. No.: US 2021/0379042 A1
(43) Pub. Date: Dec. 9, 2021

(54) METHODS FOR MONITORING TUMOR LYSIS SYNDROME

(71) Applicant: Sumitomo Dainippon Pharma Oncology, Inc., Cambridge, MA (US)

(72) Inventors: Susan Carol SMITH, Cambridge, MA (US); Stephen Patrick ANTHONY, Cambridge, MA (US)

(21) Appl. No.: 17/284,409

(22) PCT Filed: Oct. 11, 2019
(86) PCT No.: PCT/US19/55986

§ 371 Date: Apr. 9, 2021

Related U.S. Application Data
(60) Provisional application No. 62/745,269, filed on Oct. 12, 2018.
(60) Provisional application No. 62/871,799, filed on Jul. 9, 2019.

(51) Int. Cl. A61K 31/453; G01N 33/574; A61K 31/7068; A61P 35/00

(52) U.S. Cl. CPC A61K 31/453; A61P 35/00; A61K 31/7068; G01N 33/574

(57) ABSTRACT

Provided herein are methods for monitoring the development of tumor lysis syndrome (TLS) in subjects being treated for cancer with alvocidib, and methods for treating cancer using such monitoring methods. Methods for monitoring a subject for TLS can comprise performing a laboratory TLS panel on the subject about three to about four hours after the end of an alvocidib administration. Methods for treating cancer comprise administering an effective amount of alvocidib to a subject, monitoring the subject being treated with alvocidib for TLS, and administering to the subject an effective amount of one or more TLS therapies if the subject has an elevated serum potassium level or an abnormal laboratory TLS panel.

### RELATED APPLICATIONS

This application claims the benefit of U.S. Provisional Application No. 62/871,799, filed on Jul. 9, 2019, and U.S. Provisional Application No. 62/745,269, filed on Oct. 12, 2018. The entire teachings of these applications are incorporated herein by reference.

### TECHNICAL FIELD

The present disclosure relates to methods for monitoring the development of tumor lysis syndrome (TLS) in subjects being treated for cancer, and methods for treating cancer using such monitoring methods. More specifically, the subjects being monitored are suffering from hematological cancers and being treated with alvocidib.

### BACKGROUND

Tumor lysis syndrome (TLS) is a metabolic syndrome that is caused by the sudden killing of tumor cells with chemotherapy, radiotherapy, etc., or spontaneous lysis of tumors. When tumor cells die rapidly, they release their cellular contents, including large amounts of potassium, phosphate, and nucleic acids, into the systemic circulation. TLS causes hyperkalemia, hyperphosphatemia, hypocalcemia, hyperuricemia, and higher than normal levels of blood urea nitrogen (BUN) and other nitrogen-containing compounds (azotemia). Hyperuricemia and hyperphosphatemia, for example, lead to acute kidney injury and acute renal failure. In some cases, TLS leads to a reduction in the amount of chemotherapeutic agent being delivered, or cessation of the treatment until the patient recovers, which may be detrimental to the overall treatment of the cancer.

Accordingly, there is a need for methods for monitoring the development of and/or treating TLS in subjects being treated for cancer, and methods for treating cancer using such monitoring and/or treatment methods.

### BRIEF SUMMARY OF THE INVENTION

Alvocidib (flavopiridol) is a potent cyclin-dependent kinase (CDK) inhibitor with selectivity for CDKs 9, 1, 2, 4 and 7. Effects on CDK 9 may be particularly relevant to inducing apoptosis in cancers, including malignant hematopoietic cells. However, alvocidib treatment has been associated with significant side effects resulting in severe systemic reactions, including TLS. (Phelps et al., Blood, Vol. 112, (2009): 2637-2645; Karp et al., Vol. 117, (2011): 3302-3310.) The severity of TLS necessitates implementation of additional monitoring and potentially prophylactic and/or therapeutic strategies to rapidly intervene if TLS occurs. Responsiveness to the alvocidib treatment also indicates a higher susceptibility to TLS. Studies have shown that MCL-1 dependence can predict the clinical activity of alvocidib in AML patient samples and suggests an important role for MCL-1 dependence in predicting TLS risk.

Current protocols for monitoring the development of TLS in patients vary greatly due to interindividual clinician experience and different standards of care among institutions. The risk for and rapid development of TLS in cancer patients being treated with alvocidib presents a need for more precise but clinically practical TLS monitoring protocols that allow subjects to remain on alvocidib treatment and avoid dose-limiting TLS. To date, there have been no deaths attributed to TLS in clinical trials using a TLS monitoring protocol described herein.

Described herein are methods for treating hematological cancers in a subject. In one aspect, the methods comprise administering an effective amount of alvocidib to the subject, monitoring the subject for tumor lysis syndrome (TLS) by performing a serum potassium assay on the subject at the end of the alvocidib administration and about two hours after the end of the alvocidib administration, and performing a laboratory TLS panel on the subject about four hours after the end of the alvocidib administration; and administering to the subject an effective amount of one or more TLS therapies if the subject has an elevated serum potassium level or an abnormal laboratory TLS panel.

In another aspect, the methods comprise administering an effective amount of alvocidib to the subject once a day for three days; monitoring the subject for TLS by: (i) performing a serum potassium assay on the subject at the end of the alvocidib administration and about two hours after the end of the alvocidib administration on the first day, (ii) performing a laboratory TLS panel on the subject about four hours after the end of the alvocidib administration on the first day, (iii) performing an additional laboratory TLS panel on the subject about every two hours or about every four hours after the laboratory TLS panel in (ii), until twenty-four hours after the end of the alvocidib administration on the first day, and (iv) performing an additional laboratory TLS panel on the subject about every 6 hours for two days after the last laboratory TLS panel performed in (iii); and administering to the subject an effective amount of one or more TLS therapies if the subject has an elevated serum potassium level or an abnormal laboratory TLS panel.

In yet another aspect, wherein the subject is high risk for developing TLS, the methods comprise administering an effective amount of alvocidib to the subject (e.g., once a day for three days); monitoring the subject for TLS by: (i) performing a serum potassium assay on the subject at the end of the alvocidib administration and about two hours after the end of the alvocidib administration, (ii) performing a laboratory TLS panel on the subject about four hours after the end of the alvocidib administration, (iii) performing an additional laboratory TLS panel on the subject about every two hours after the laboratory TLS panel in (ii), until twenty-four hours after the end of the alvocidib administration, and (iv) performing an additional laboratory TLS panel on the subject about every 6 hours for two days after the last laboratory TLS panel performed in (iii); and administering to the subject an effective amount of one or more TLS therapies if the subject has an elevated serum potassium level or an abnormal laboratory TLS panel.

In another aspect, wherein the subject is not high risk for developing TLS, the methods comprise administering an effective amount of alvocidib to the subject (e.g., once a day for three days); monitoring the subject for TLS by: (i) performing a serum potassium assay on the subject at the end of the alvocidib administration and about two hours after the end of the alvocidib administration, (ii) performing a laboratory TLS panel on the subject about four hours after the end of the alvocidib administration, (iii) performing an additional laboratory TLS panel on the subject about every four hours after the laboratory TLS panel in (ii), until twenty-four hours after the end of the alvocidib administration, and (iv) performing an additional laboratory TLS panel on the subject about every 6 hours for two days after the last laboratory TLS panel performed in (iii); and administering to the subject an effective amount of one or more TLS therapies if the subject has an elevated serum potassium level or an abnormal laboratory TLS panel.

In another aspect, wherein the AML, is frontline, relapsed or refractory (e.g., relapsed or refractory), the methods comprise administering an effective amount of alvocidib to the subject; monitoring the subject for TLS by performing a serum potassium assay on the subject at the end of the alvocidib administration and about two hours after the end of the alvocidib administration, and performing a laboratory TLS panel on the subject about four hours after the end of the alvocidib administration; and administering to the subject an effective amount of one or more TLS therapies if the subject has an elevated serum potassium level or an abnormal laboratory TLS panel. If the subject exhibits evidence of clinically meaningful TLS after the alvocidib administration, an additional laboratory TLS panel is performed on the subject about every two hours after the laboratory TLS panel, until twenty-four hours after the end of the alvocidib administration. If the subject does not exhibit evidence of clinically meaningful TLS after the alvocidib administration, an additional laboratory TLS panel is performed on the subject about every four hours after the laboratory TLS panel, until twenty-four hours after the end of the alvocidib administration.

In another aspect, the methods comprise administering an effective amount of alvocidib to the subject; monitoring the subject for TLS by performing a laboratory TLS panel on the subject from three to about four hours after the end of the alvocidib administration, and performing an additional laboratory TLS panel on the subject daily for three days following the alvocidib administration; and administering to the subject an effective amount of one or more TLS therapies if the subject has an abnormal laboratory TLS panel.

In another aspect, the methods comprise administering a prophylactically effective amount of each of: intravenous (IV) hydration, allopurinol and an oral phosphate binder to the subject; administering a therapeutically effective amount of alvocidib to the subject; monitoring the subject for TLS by performing a laboratory TLS panel on the subject about three to about four hours after the end of the alvocidib administration; and administering to the subject a therapeutically effective amount of one or more TLS therapies if the subject has an abnormal laboratory TLS panel.

Also described herein are methods for reducing the severity of TLS in a hematological cancer subject being treated with alvocidib, reducing the incidence of TLS in hematological cancer subjects being treated with alvocidib, monitoring for TLS while treating a hematological cancer in a subject with alvocidib, decreasing mortality from TLS in hematological subjects being treated with alvocidib, increasing survival time of hematological cancer subjects being treated with alvocidib, treating TLS in a hematological cancer subject being treated with alvocidib, preventing TLS in a hematological cancer subject being treated with alvocidib, and/or diagnosing and treating TLS in a hematological cancer subject being treated with alvocidib. In one aspect, the methods comprise monitoring the subject for TLS by performing a serum potassium assay on the subject at the end of the alvocidib administration and about two hours after the end of the alvocidib administration; and performing a laboratory TLS panel on the subject about four hours after the end of the alvocidib administration; and administering to the subject an effective amount of one or more TLS therapies if the subject has an elevated serum potassium level or an abnormal laboratory TLS panel.

In another aspect, the methods comprise monitoring the subject for TLS by performing a laboratory TLS panel on the subject from three to about four hours after the end of the alvocidib administration, and performing an additional laboratory TLS panel on the subject daily for three days following the alvocidib administration; and administering to the subject an effective amount of one or more TLS therapies if the subject has an abnormal laboratory TLS panel.

In another aspect, the methods comprise administering a prophylactically effective amount of each of: IV hydration, allopurinol and an oral phosphate binder to the subject; monitoring the subject for TLS by performing a laboratory TLS panel on the subject about three to about four hours after the end of the alvocidib administration; and administering to the subject a therapeutically effective amount of one or more TLS therapies if the subject has an abnormal laboratory TLS panel.

In another aspect, the method is a method of treating TLS in a hematological cancer subject being treated with alvocidib, and comprises monitoring the subject for TLS by performing a serum potassium assay on the subject at the end of the alvocidib administration and about two hours after the end of the alvocidib administration, and performing a laboratory TLS panel on the subject about four hours after the end of the alvocidib administration; determining the subject has an elevated serum potassium level or an abnormal laboratory TLS panel; and administering to the subject an effective amount of one or more TLS therapies.

In another aspect of a method of treating TLS in a hematological cancer subject being treated with alvocidib, the method comprises monitoring the subject for TLS by performing a laboratory TLS panel on the subject about three to about four hours after the end of the alvocidib administration; determining the subject has an abnormal laboratory TLS panel; and administering to the subject an effective amount of one or more TLS therapies.

### DETAILED DESCRIPTION OF THE INVENTION

The following paragraphs define in more detail the embodiments of the invention described herein. The following embodiments are not meant to limit the invention or narrow the scope thereof, as it will be readily apparent to one of ordinary skill in the art that suitable modifications and adaptations may be made without departing from the scope of the invention, embodiments, or specific aspects described herein. All patents and publications cited herein are incorporated by reference herein in their entirety.

As used herein, “a” or “an” means one or more unless otherwise specified.

Terms such as “include,” “including,” “contain,” “containing,” “having,” and the like mean “comprising.”

As used herein, the terms “dosage” or “dose” or “dosage form” denote any form or formulation of a therapeutic agent that contains an amount sufficient to produce a therapeutic effect with a single administration.

As used herein, the term “about,” with respect to a particular time or period of time, such as “about a number of hours,” means a time point or time period that is more than, less than, and including that particular time or time period. In one embodiment, “about” with respect to a particular time or time period means a thirty-minute variance, such as thirty minutes after or more than, thirty minutes before or less than, and including the particular time or time period. For example, the phrase “about two hours” means a time period that is one and one-half hours to two and one-half hours in length, or a time point that occurs at any time in between one and one-half hours and two and one-half hours, including one and one-half and two and one-half hours. If particularly stated, “about” with respect to a particular time or time period, means a variance of an amount of time that is less than thirty minutes, for example, twenty minutes, fifteen minutes, ten minutes, five minutes, or even two minutes or one minute. As such, the term “about two hours,” when a specific reference to a fifteen-minute time variance is made, means one and three-quarter hours to two and one quarter hours.

The term “about” has its ordinary meaning when used in any other context that is not in reference to a particular time or time period.

The monitoring methods of the present disclosure include performing one or more assays or tests on a subject (e.g., serum potassium assay, laboratory TLS panel, lactic acid dehydrogenase (LDH) assay, fibrinogen assay). These assays and tests are well understood to those skilled in the art and can vary in method from institution to institution, and/or laboratory to laboratory. Generally, tests or assays, such as serum potassium assays or laboratory TLS panels, comprise obtaining or having obtained a biological sample from a subject, and preserving or manipulating the sample in such a way that a quantitative or qualitative chemical or biological assay can be completed to determine the amount or identity of a particular substance in the sample.

As used herein, the term “serum potassium assay” refers to a diagnostic assay that measures the concentration of potassium electrolyte in the serum portion of a subject's blood.

As used herein, the term “elevated serum potassium” means a concentration of potassium ions in the serum portion of a subject's blood that is greater than the concentration that is considered within normal limits. While it is often understood that concentrations of 3.5 mEq/L-5.0 mEq/L are within normal limits, in certain embodiments, elevated serum potassium levels are those that are greater than 4.0 mEq/L, as well as those that are greater than 5.0 mEq/L, or greater than 5.5 mEq/L. In another embodiment, elevated serum potassium levels are levels that are at least 25% above the subject's baseline level.

Typically, samples (e.g., blood, serum samples) used to determine baseline levels of a subject's analyte(s) are collected prospectively (e.g., prior to administration of alvocidib). Typically, a baseline level will be determined from a laboratory TLS panel performed prior to and within seven days (e.g., five days, three days, two days, 24 hours, 12 hours, 6 hours, 4 hours, 2 hours, 1 hour, 30 minutes) of the start of treatment in accordance with the methods described herein (e.g., the start of the alvocidib administration, particularly the first administration of alvocidib, if treatment includes more than one dose of alvocidib). In some embodiments, samples used to determine baseline levels are collected within 7 days of the start of treatment in accordance with the methods described herein. In some embodiments, samples used to determine baseline levels are collected within 3 days of the start of treatment in accordance with the methods described herein. In some embodiments, samples used to determine baseline levels are collected on day 1 of the treatment in accordance with the methods described herein, preferably, prior to the start of treatment (e.g., administration of alvocidib).

As used herein, the term “laboratory TLS panel” means at least two diagnostic tests that are utilized alone or in combination to diagnose a subject for the presence of TLS, or provide evidence of clinically meaningful TLS in a subject. The specific diagnostic tests that comprise a laboratory TLS panel can vary from institution to institution, but typically include one or more of the following diagnostic assays: serum phosphate assay for detecting hyperphosphatemia; serum uric acid assay for detecting hyperuricemia; serum electrolyte assays including serum sodium assay for detecting hypernatremia, serum potassium assay for detecting hyperkalemia, serum chloride assay for detecting hyperchloremia, and serum carbon dioxide assay for detecting acidosis or alkalosis; serum calcium assay for detecting hyper- or hypocalcemia; serum creatinine assay for detecting renal injury or failure; and serum lactate dehydrogenase (LDH) assay for detecting tissue damage. In one embodiment, the laboratory TLS panel comprises a serum potassium assay, a serum uric acid assay, a serum chloride assay, a serum sodium assay, a serum creatinine assay, a serum phosphate assay, a serum calcium assay, a serum LDH assay, and a serum carbon dioxide assay. In another embodiment, the laboratory TLS panel comprises a serum potassium assay, a serum uric acid assay, a serum phosphate assay, and a serum calcium assay. In another embodiment, a laboratory TLS panel comprises a serum potassium assay. In yet another embodiment, a laboratory TLS panel comprises a serum phosphate assay for detecting hyperphosphatemia; serum uric acid assay for detecting hyperuricemia; serum electrolyte assays including serum sodium assay for detecting hypernatremia, serum potassium assay for detecting hyperkalemia, serum chloride assay for detecting hyperchloremia, and serum carbon dioxide assay for detecting acidosis or alkalosis; serum calcium assay for detecting hyper- or hypocalcemia and serum creatinine assay for detecting renal injury or failure. In yet other embodiments, a laboratory TLS panel comprises a serum calcium assay; serum phosphate assay; serum potassium assay; serum uric acid assay; serum LDH assay; and serum creatinine assay.

As used herein, the term “abnormal laboratory TLS panel” means (i) at least two of the results from the diagnostic tests of potassium, uric acid, phosphate and calcium show greater than 25% change from baseline values or above the normal laboratory values, or (ii) at least one of the diagnostic tests of potassium, uric acid, phosphate and calcium is above normal limits and serum creatinine levels are above 1.4 mg/dL. Normal limits can vary among institutions, however, the skilled artisan can readily recognize normal laboratory values or above normal laboratory values. For example, a serum level of potassium greater than 5 mEq/L, uric acid greater than 7.5 mg/dL, phosphate greater than 5 mg/dL and/or calcium less than 8 mg/dL can all be indicative of serum levels above normal limits.

Methods of performing, processing and/or certifying serum assays, such as a serum potassium assay, or an assay included in a laboratory TLS panel, are well-known in the art. Often, hospitals have automated laboratory assay machines that run the assays.

As used herein, the term “clinically meaningful TLS” means one or more of hyperkalemia, hyperuricemia, hyperphosphatemia, increased lactate dehydrogenase (LDH), coagulopathy, and cytokine release syndrome, with a degree of severity that therapeutic intervention is indicated and/or required. In certain embodiments, “clinically meaningful TLS” includes clinical TLS. “Clinical TLS” means the presence of an abnormal laboratory TLS panel and at least one of the following TLS-related complications in the absence of any other recognizable cause, that requires treatment: oliguric renal failure (urine output <800 mL/day), requirement for hemodialysis, electrocardiographic signs of hyperkalemia, cardiac arrhythmia/sudden death, tetany or seizures. In certain embodiments, “clinically meaningful TLS” includes laboratory TLS. “Laboratory TLS” means the presence of an abnormal laboratory TLS panel. In certain embodiments, clinically meaningful TLS is grade 3, grade 4 and/or grade 5 TLS, according to CTCAE 5.0 TLS grading.

As used herein, the terms “prevention of,” “prevent” and “preventing” include reducing progression of a disorder, such as cancer or TLS, either to a statistically significant degree or to a degree detectable to one skilled in the art. The amount of the reduction may be from about 0.01% to about 100%. In some embodiments, the term preventing refers to the prevention of TLS (e.g., grade 3, grade 4 and/or grade 5 TLS, according to CTCAE 5.0 TLS grading).

As used herein, the terms “treat” and “treating” refers to administering a therapy to a subject with the purpose (e.g., in an amount, manner, or mode effective, for example, therapeutically effective) to improve a condition, symptom, disorder, or parameter associated with a disorder, or a likelihood thereof. In some embodiments, the term treating refers to the treatment of a cancer, such as a hematological cancer. In other embodiments, the term treating refers to the treatment of TLS in a subject (e.g., patient).

The methods of the present disclosure comprise administration of alvocidib (e.g., an effective amount of alvocidib) to a subject. As used herein, the term “alvocidib” means 2-(2-chlorophenyl)-5,7-dihydroxy-8-[(3 S,4R)-3-hydroxy-1-methylpiperidin-4-yl]chromen-4-one, or a pharmaceutically acceptable salt thereof (e.g., 2-(2-chlorophenyl)-5,7-dihydroxy-8-[(3S,4R)-3-hydroxy-1-methylpiperidin-4-yl]chromen-4-one hydrochloride).

The method of the present disclosure can alternatively comprise administration of a prodrug of alvocidib, or a pharmaceutically acceptable salt thereof (e.g., an effective amount of a prodrug of alvocidib, or a pharmaceutically acceptable salt thereof), to a subject. Even if not specifically described in each instance, all embodiments which include alvocidib, optionally comprise use of a prodrug of alvocidib instead of, or in addition to (typically, instead of), alvocidib.

“Prodrug” is meant to indicate a compound that may be converted under physiological conditions or by solvolysis to a biologically active compound. Thus, the term “prodrug” refers to a precursor of a biologically active compound that is pharmaceutically acceptable. In some aspects, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein for their teachings regarding the same. The term “prodrug” is also meant to include any covalently bonded carriers, which release the active compound in vivo when such prodrug is administered to a subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound and the like.

Examples of prodrugs of alvocidib are described in International Publication Nos. WO 2016/187316 and WO 2018/094275, which are incorporated herein by reference in their entireties for their teachings regarding the same. In some embodiments, the prodrug of alvocidib is a phosphate prodrug of alvocidib. In some instances, the prodrug of alvocidib can be a compound of structural formula I:

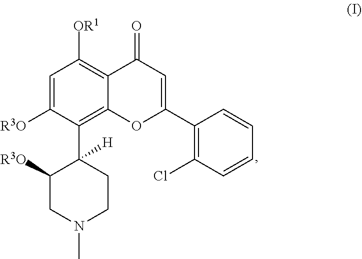

or a pharmaceutically acceptable salt thereof, wherein one of R¹, R² and R³ is —P(═O)(OH)₂, and the other two of R¹, R² and R³ are each —H. In some instances, the prodrug of alvocidib can be the compound of structural formula Ia:

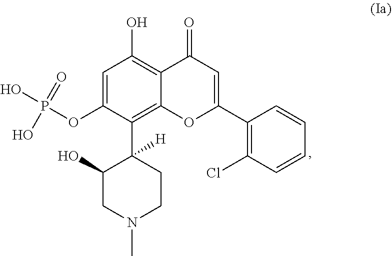 [SMILES: CN1CC[C@H](c2c(OP(=O)(O)O)cc(O)c3c(=O)cc(-c4ccccc4Cl)oc23)[C@H](O)C1]

or a pharmaceutically acceptable salt thereof. The compounds of Structural Formulas I and Ia are orally bioavailable. Thus, the compounds of Structural Formulas I and Ia, or a pharmaceutically acceptable salt of the foregoing, can be administered orally, and compositions comprising a compound of Structural Formula I or Ia, or a pharmaceutically acceptable salt thereof, can be formulated for oral administration.

It will be appreciated that the therapeutic and prophylactic agents disclosed herein can be present as free bases, free acids and/or pharmaceutically acceptable salts. When a therapeutic or prophylactic agent is identified herein, such identification includes the therapeutic or prophylactic agent in free base or free acid form, or as a pharmaceutically acceptable salt, unless otherwise indicated. For example, identification of allopurinol includes allopurinol, or a pharmaceutically acceptable salt of allopurinol, unless otherwise indicated. In some embodiments, the identified agent is present as a free base or free acid. In some embodiments, the identified agent is present as a pharmaceutically acceptable salt.

As used herein, the term “pharmaceutically acceptable salt” refers to those salts that are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. Pharmaceutically acceptable salts of the compounds described herein include those derived from suitable inorganic and organic acids and bases.

Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “effective amount” is an amount that achieves the desired effect (e.g., prevention and/or treatment of cancer and/or TLS). For example, in the present invention, an effective amount of alvocidib, after one or more administrations, alone or in combination with or in series with additional therapeutic agents, causes a beneficial or therapeutic effect in the subject receiving alvocidib. Typically, an effective amount of alvocidib is an amount of alvocidib, after one or more administrations, alone or in combination with or in series with additional therapeutic agents, that achieves treatment of cancer (e.g., AML), and an effective amount of a TLS therapy is an amount of the TLS therapy, after one or more administrations, alone or in combination with or in series with additional therapeutic agents, that achieves treatment or prevention (e.g., prevention, in the case of TLS therapies that are administered prophylactically) of TLS.

As used herein, a “therapeutically effective amount” is an amount that achieves treatment (e.g., improvement in a condition, symptom, disorder, or parameter associated with a disorder, or a likelihood thereof, either to a statistically significant degree or to a degree detectable to one skilled in the art) of a disease or condition (e.g., a hematological cancer, TLS). In some embodiments, a therapeutically effective amount of alvocidib is administered to a subject. In some embodiments, particularly in those where a TLS therapy is being administered to a subject because the subject has an elevated serum potassium level and/or an abnormal laboratory TLS panel, a therapeutically effective amount of one or more TLS therapies is administered to a subject.

As used herein, a “prophylactically effective amount” is an amount that achieves prevention of a disease or condition (e.g., TLS). In some embodiments, a prophylactically effective amount of one or more TLS therapies (e.g., IV hydration, allopurinol, an oral phosphate binder) is administered to a subject. It will be understood by the skilled clinician that the therapeutically effective amount and the prophylactically effective amount of an agent need not be different in the context of this invention, though they can be.

In one embodiment, an effective amount of alvocidib is administered to the subject as a 30-minute IV bolus of alvocidib followed by a 4-hour IV infusion of alvocidib. In certain embodiments, the 30-minute IV bolus is 30 mg/m² of alvocidib. In further embodiments, the 4-hour IV infusion is 60 mg/m² of alvocidib. An effective amount of alvocidib can be administered to a subject for one day, or for a number of days, including two days, or three days. In one embodiment, the effective amount of alvocidib is a 30-minute IV bolus of 30 mg/m² of alvocidib, followed by a 4-hour IV infusion of 60 mg/m² of alvocidib, administered to a subject for three consecutive days. In one embodiment, the effective amount of alvocidib is a 30-minute IV bolus of 30 mg/m² of alvocidib, followed by a 4-hour IV infusion of 60 mg/m² of alvocidib, administered to a subject once per treatment cycle.

When a dose of alvocidib comprising a bolus and an infusion is administered to a subject, for example, using a bolus-infusion dose and/or schedule described herein, a “hybrid” dose of alvocidib is administered. Thus, in some embodiments, an effective amount of alvocidib is administered to a subject by one or more hybrid doses. In some embodiments, a subject is being treated with alvocidib administered by one or more hybrid doses.

In one embodiment, an effective amount of alvocidib is administered to the subject as a 30-60-minute IV bolus. In certain embodiments, the 30-60-minute IV bolus is from about 25 mg/m² to about 50 mg/m² (e.g., about 25 mg/m², about 50 mg/m²) alvocidib. In one embodiment, the effective amount of alvocidib is a 30-60-minute IV bolus of about 25 mg/m² alvocidib, administered on day 1 of a treatment cycle (e.g., 28-day treatment cycle), and a 30-60-minute IV bolus of about 50 mg/m² alvocidib, administered on days 8 and 15 of a treatment cycle (e.g., 28-day treatment cycle).

In some embodiments, an effective amount of a prodrug of alvocidib (e.g., a compound of Structural Formula I or Ia), or a pharmaceutically acceptable salt thereof, is administered to a subject orally, for example, in a dose of about 1 mg or 2 mg twice a day, or about 1 mg or about 2 mg once a day. An effective amount of a prodrug of alvocidib can be administered to a subject for one day, two days, three days, four days, five days, six days, seven days, two weeks, three weeks, four weeks, two months, three months, four months, five months, etc.

Additional cytotoxic drugs can be administered in combination with or in series with alvocidib. Those skilled in the art readily recognize the one or more cytotoxic drugs that are useful for treating hematological cancers in combination with alvocidib. In some embodiments, the subject is administered at least one cytotoxic drug, or at least two cytotoxic drugs. As used herein, the term “cytotoxic drug” refers to a medicine that contains a chemical that is toxic to cells, preventing their replication, growth or survival. Examples of cytotoxic drugs include cytarabine, mitoxantrone, daunorubicin, idarubicin, hypomethylating agents such as decitabine and azacytidine, and venetoclax.

In one embodiment, the subject is administered an effective amount of cytarabine 5 days after alvocidib is first administered. In one embodiment, the subject is administered an effective amount of cytarabine 1 day after alvocidib is administered, e.g., over a period of 10 days, beginning on the first day after alvocidib is administered. For example, cytarabine is administered once daily for 10 days, beginning 1 day after alvocidib is administered. In some embodiments, the subject is administered an effective amount of cytarabine on the second day after alvocidib is administered, e.g., over a period of ten days beginning on the second day after alvocidib is administered.

The effective amount of cytarabine can be determined by the skilled artisan based upon a subject's body weight, disease state, and various other factors specific to the subject. In certain embodiments, the effective amount of cytarabine is administered as a continuous IV infusion for about 72 hours in an amount of 667 mg/m² per 24 hours, for a total of 2 gm/m². In certain embodiments, the effective amount of cytarabine is administered as a subcutaneous injection, e.g., in an amount of about 20 mg/m² per day. In certain embodiments, the effective amount of cytarabine is administered as a subcutaneous injection once daily for ten days in an amount of 20 mg/m² per day, for a total of 200 mg/m².

In another embodiment, the subject is administered an effective amount of mitoxantrone, e.g., about 12 hours after the end of the cytarabine administration. In some embodiments, the effective amount of mitoxantrone is administered as an IV infusion over about 1 to 2 hours in an amount of 40 mg/m².

In certain embodiments, the subject is administered an effective amount of idarubicin or daunorubicin, and an effective amount of cytarabine. For example, the subject is administered from about 45 mg/m² to about 110 mg/m² (e.g., about 60 mg/m²) daunorubicin per day, administered by intravenous bolus of from 5 minutes to 30 minutes±15 minutes in duration, e.g., on the fifth, sixth and seventh days of a treatment cycle. The subject is administered from about 90 mg/m² to about 110 mg/m² (e.g., about 100 mg/m²) cytarabine per day, administered by intravenous infusion of from about 20 hours to about 28 hours in duration on the fifth, sixth, seventh, eighth, ninth, tenth, and eleventh days of the treatment cycle.

In one embodiment, the subject (e.g., a subject with MDS) is administered an effective amount of a hypomethylating agent (e.g., decitabine, azacytidine) in combination with or in series with alvocidib. For example, the subject is administered decitabine (e.g., about 20 mg/m² decitabine via IV infusion of, for example, about one hour) daily on days 1-5 of a treatment cycle (e.g., a 28-day treatment cycle). Alternatively, the subject is administered azacytidine (e.g., about 75 mg/m² or about 100 mg/m² via subcutaneous injection or intravenous infusion) daily on days 1-7 of a treatment cycle (e.g., a 28-day treatment cycle). On day 8, the subject is administered alvocidib (e.g., a hybrid dose of alvocidib, such as about 30 mg/m² alvocidib as an about 30-minute bolus±15 minutes followed by 60 mg/m² as an about four-hour IV infusion). In some embodiments, the subject is administered about 75 mg/m² azacytidine via subcutaneous injection or intravenous infusion daily on days 1-7 of a first and/or second (e.g., first) treatment cycle (e.g., a 28-day treatment cycle), and about 100 mg/m² azacytidine via subcutaneous injection or intravenous infusion daily on days 1-7 of one or more (e.g., 2, 3, 4, 5, 6, 7, 8, 9, 10, 11, 12) subsequent treatment cycles.

In some embodiments of the methods disclosed herein, more than one cycle of treatment is necessary. Accordingly, the methods disclosed herein can include one or more treatment cycles, e.g., 1, 2, 3, 4, 5, 6, 7, 8, 9, 10, 11, 12, etc. treatment cycles. The length of a treatment cycle is determined by the treatment being administered, but can be 1, 2, 3, 4, 5, 6, 7, 8, 9, 10, 11, 12, 13, 14, 15, 16, 17, 18, 19, 20, 21, 22, 23, 24, 25, 26, 27 or 28 days, or 4, 5, 6, 7, 8, 9, 10, 11 or 12 weeks. The appropriate length of a treatment cycle can be determined by a clinician skilled in the art.

Studies have shown that MCL-1 dependence can predict the clinical activity of alvocidib in AML patient samples, and suggest an important role for MCL-1 activity in predicting alvocidib activity. See, for example, International Publication Nos. WO 2016/172214 and WO 2018/119000, incorporated herein by reference to the extent that MCL-1 activity is predictive of alvocidib activity in AML patients. In one embodiment of the invention, the subject with hematological cancer is also MCL-1 dependent. The term “MCL-1 dependency,” as used herein, means a hematological cancer that primarily or solely utilizes the protein, MCL-1, to suppress normal signals leading to the induction of apoptosis. MCL-1 dependency is most commonly determined by the depolarization of mitochondrial membrane potential due to an MCL-1-targeted BH3 peptide. In one embodiment, the subject has an MCL-1 dependence score selected from greater than or equal to 40%, 30% to less than 40%, 15% to less than 30%, and 0 to less than 15%.

As used herein, the term “subject” refers to any mammal, including animals and humans. The subject may be a cancer patient or, more specifically, a hematological cancer patient, in need of treatment. In certain embodiments, the subject (e.g., patient) is less than or equal to 65 years of age.

As used herein, the term “patient” refers to a human subject.

The term “substantially,” as used herein, means to a great or significant extent, but not completely.

The Common Terminology Criteria for Adverse Events (CTCAE) 5.0 describes tumor lysis syndrome (TLS) as a disorder characterized by metabolic abnormalities that result from a spontaneous or therapy-related cytolysis of tumor cells. CTCAE 5.0 grades TLS as grade 3 if the syndrome is present, as grade 4 if the syndrome is associated with life-threatening consequences and/or urgent intervention is indicated, and grade 5 if the syndrome results in death. In some embodiments, CTCAE 5.0 criteria are used to diagnose and/or identify the presence of TLS in a subject.

As used herein, the term “TLS therapy” refers to a treatment for hyperkalemia, hyperuricemia, hyperphosphatemia, coagulopathy, increased serum creatinine, cytokine release syndrome, oliguric renal failure (e.g., urine output less than 800 mL/day), cardiac arrhythmia, tetany and/or seizures. Such treatments are administered to a patient in response to the development of one or more of the aforementioned conditions or disorders, and such treatments can be administered once, or multiple times to a subject. TLS therapies include, but are not limited to, prophylactic administration of pretreatment IV hydration, oral allopurinol, and oral phosphate binder, as well as diligent monitoring of urine output to ensure that it equals fluid input. If input is greater than output by 10%, administration of diuretics is recommended. Replacement of excessive fluid losses, including from diarrhea is also recommended, unless otherwise clinically indicated, along with the following treatments related to laboratory abnormalities:
If potassium levels are increasing to >4.0 mEq/L, patients should receive a 30-gm dose of sodium polystyrene sulfonate, unless there are other likely causes of hyperkalemia other than TLS, or a contraindication to its use.
If potassium levels rise to >5.0 mEq/L, in addition to the 30-gm dose of sodium polystyrene sulfonate, patients should also receive 10 units of IV rapid-acting insulin and 25 gm (one ampoule) of IV dextrose 50%, unless there are other likely causes of hyperkalemia other than TLS, or a contraindication to its use.
If potassium levels rise to >5.5 mEq/L, patients should be considered for emergent intermittent or continuous dialysis.
Calcium supplementation should only be given for symptomatic hypocalcemia in this setting to avoid renal precipitation of calcium phosphate crystals.
Patients who develop clinical evidence of cytokine release syndrome or who have hyperkalemia requiring dialysis will receive immediate steroid therapy with an equivalent of at least 20 mg of IV dexamethasone.

Subjects with hematological cancers can have varying levels of risk for developing TLS when being treated with alvocidib. In certain embodiments, the subject is at high risk for developing TLS. In other embodiments, the subject is at low or moderate risk for developing TLS. There are a number of ways for determining the level of risk any particular subject has with respect to developing TLS, although certain scoring systems are generally recognized by those skilled in the art. For example, a multivariate analysis and risk score prediction model for the development of TLS in AML patients is summarized in Table 1. Subjects with an overall score of greater than or equal to 2 are considered at high risk for development of TLS. Subjects with an overall score of 1 are considered at moderate risk for developing TLS. Subjects with an overall score of 0 are considered at low risk for development of TLS.

TABLE 1

| | | Regres- | CTLS | | | |
|---|---|---|---|---|---|---|
| Co- | Unfavorable | sion co- | Odds ratio | | p | |
| variate | categories | efficient | (95% CI) | | value | Score |
| WBC | ≤25 × 10⁹/L | 1.1 | 1 | | <0.001 | 0 |
| | 25.75 × 10⁹/L | | 2.7 | (1.4-5.4) | | 1 |
| | >75 × 10⁹/L | | 7.3 | (2.0-29.1) | | 2 |
| Uric | ≤7.5 mg/dL | 2.2 | 1 | | <0.001 | 0 |
| acid | >7.5 mg/dL | | 9.1 | (3.3-26.6) | | 1 |
| LDH | ≤1 × ULN | 1.2 | 1 | | 0.005 | 0 |
| | 1.4 × ULN | | 3.9 | (1.5-10.8) | | 1 |
| | >4 × ULN | | 15.2 | (2.2-96.8) | | 2 |

CTLS = clinical tumor lysis syndrome;
LDH = lactate dehydrogenase;
ULN = upper limit of normal;
WBC = white blood cells
Montesinos P, Lorenzo I, Martin G, et al. Haematologica 2008; 93(1): 67-74.

Differing treatment regimens (e.g., intensive induction chemotherapy versus non-intensive induction chemotherapy; intravenous administration versus oral administration) can also create varying levels of risk for developing TLS when being treated with alvocidib. Thus, some embodiments comprise administering alvocidib to a subject, wherein the alvocidib is being administered as part of intensive induction chemotherapy (e.g., as when a subject is being treated with intensive induction chemotherapy comprising alvocidib). Some embodiments comprise intravenously administering alvocidib to a subject (e.g., as when a subject is being treated with intravenous alvocidib, in particular, a hybrid dose of intravenous alvocidib comprising a bolus and an infusion).

Examples of intensive induction chemotherapies include the therapies described in Examples 1 and 2 herein.

A number of hematological cancers can be treated with alvocidib using the methods of the present invention. As used herein, the term “hematological cancers” means a cancer that begins in blood-forming tissue, such as the bone marrow, or in the cells of the immune system. Examples of hematologic cancer are leukemia, lymphoma, and multiple myeloma. In some embodiments, a hematologic cancer is multiple myeloma, myelodysplastic syndrome (MDS), acute myeloid leukemia (AML), acute lymphoblastic leukemia (ALL), acute lymphocytic leukemia, chronic lymphogenous leukemia, chronic lymphocytic leukemia (CLL), mantle cell lymphoma, diffuse large B-cell lymphoma, follicular lymphoma, or non-Hodgkin's lymphoma. In one embodiment, the hematological cancer is acute myeloid leukemia (AML). In a further embodiment, the AML is refractory AML or relapsed AML (e.g., AML refractory to or relapsed from a prior treatment comprising venetoclax, for example, venetoclax in combination with azacytidine or decitabine). In another embodiment, the AML is frontline AML. In other embodiments, the AML is primary AML. In other embodiments, the hematological cancer is myelodysplastic syndrome (MDS). In other embodiments, the hematological cancer is multiple myeloma.

As used herein, the term “refractory” and “resistant” are used interchangeably, and each refers to a disease, such as cancer (e.g., AML) that does not respond to a treatment, either because the cancer is resistant to the treatment at the outset of the treatment or becomes resistant to the treatment during treatment. In some embodiments, “refractory” means a subject failed to achieve CR following treatment for a disease, or achieved a CR lasting less than 90 days following treatment for the disease.

As used herein, the term “relapse” refers to the return of a disease, such as cancer (e.g., AML), or the signs and symptoms of the disease after a period of complete remission. In some embodiments, relapse may refer to the recurrence of disease after complete remission meeting one or more of the following criteria (i) greater than or equal to 5% blasts in the marrow or peripheral blood, and/or (ii) extramedullary disease, and/or disease (e.g., cancer) presence determined by a physician upon clinical assessment. In some embodiments, “relapse” refers to reoccurrence of a disease following a CR lasting 90 days or longer.

“Frontline” and “newly diagnosed,” used to describe a cancer herein, such as AML, means that the cancer has not previously been treated with a traditional therapy, such as radiation, surgery or chemotherapy. Accordingly, in some embodiments of the methods described herein, the cancer (e.g., hematologic cancer, such as AML) is previously untreated.

As used herein, the term “primary,” with respect to a cancer, has its ordinary meaning in the art, and may refer to the original, or first, cancer in the body.

The methods of the present disclosure include methods of treating a hematological cancer in a subject, comprising administering an effective amount of alvocidib to the subject, monitoring the subject for TLS by performing a laboratory TLS panel on the subject about three to about four hours after the end of the alvocidib administration, and administering to the subject one or more TLS therapies if the subject has an abnormal laboratory TLS panel.

The methods of the present disclosure also include methods of treating a hematological cancer in a subject, comprising administering an effective amount of alvocidib to the subject; monitoring the subject for TLS by performing a serum potassium assay on the subject at the end of the alvocidib administration and about two hours after the end of the alvocidib administration, and performing a laboratory TLS panel on the subject about four hours after the end of the alvocidib administration; and administering to the subject one or more TLS therapies if the subject has an elevated serum potassium level or an abnormal laboratory TLS panel.

In certain embodiments, the monitoring further comprises performing an additional laboratory TLS panel on the subject about every two hours after the laboratory TLS panel performed about four hours after the end of the alvocidib administration, until twenty-four hours after the end of the alvocidib administration.

In other embodiments, the monitoring further comprises performing an additional laboratory TLS panel on the subject about every four hours after the laboratory TLS panel performed about four hours after the end of the alvocidib administration, until twenty-four hours after the end of the alvocidib administration.

In some embodiments, the monitoring further comprises performing an additional laboratory TLS panel on the subject daily for three days following the alvocidib administration. In some embodiments, the monitoring further comprises performing an additional laboratory TLS panel on the subject weekly after the first week following the alvocidib administration. In some embodiments, the monitoring further comprises performing a laboratory TLS panel on the subject prior to the alvocidib administration (e.g., to obtain a subject's baseline levels).

In some embodiments, the laboratory TLS panel includes a serum potassium assay, and the method further comprises administering to the subject an effective amount of one or more TLS therapies (e.g., for an elevated serum potassium level) if the subject has an elevated serum potassium level.

Typically, each laboratory TLS panel (e.g., the initial laboratory TLS panel, the additional laboratory TLS panel, the pre-alvocidib laboratory TLS panel) will comprise the same at least two diagnostic tests. However, each laboratory TLS panel need not be identical, and differences between TLS panels are encompassed. When laboratory TLS panels are different, at least one of the two or more diagnostic tests of the laboratory TLS panels will typically be common across the laboratory TLS panels, such that the evolution of a specific TLS indicator(s) can be observed over time. In some embodiments, there will be adequate overlap between laboratory TLS panels to determine whether a subject's laboratory TLS panel is abnormal based on the subject's baseline levels.

Some embodiments further comprise terminating administration of alvocidib and/or a cytotoxic drug(s) being administered in combination or in series with alvocidib if the subject has an elevated serum potassium level and/or an abnormal laboratory TLS panel. Administration of alvocidib and/or a cytotoxic drug(s) being administered in combination or in series with alvocidib can be continued upon resolution of the TLS (e.g., as indicated by a laboratory TLS panel that is not abnormal and/or a serum potassium level that is not elevated).

Also described herein are methods for reducing the severity of TLS (e.g., from grade 5 to grade 3 or 4, from grade 4 or 5 to grade 3, eliminating grade 3, 4 or 5 TLS, according to the CTCAE 5.0 grading scale for TLS; reducing the severity of the symptoms of TLS) in a hematological cancer subject(s), reducing the incidence of TLS (e.g., grade 3 TLS, grade 4 TLS, grade 5 TLS, grade 4 or grade 5 TLS, grade 3, 4 or 5 TLS, according to the CTCAE 5.0 grading scale for TLS) in a hematological cancer subject(s) being treated with alvocidib, monitoring for TLS while treating a hematological cancer subject(s) with alvocidib, decreasing mortality from TLS in a hematological subject(s) being treated with alvocidib, increasing survival time of a hematological cancer subject(s) being treated with alvocidib, treating TLS in a hematological cancer subject(s) being treated with alvocidib, preventing TLS (e.g., grade 3 TLS, grade 4 TLS, grade 5 TLS, grade 4 or grade 5 TLS, grade 3, 4 or 5 TLS, according to the CTCAE 5.0 grading scale for TLS) in a hematological cancer subject(s) being treated with alvocidib, and/or diagnosing and/or treating TLS in a hematological cancer subject(s) being treated with alvocidib. In one aspect, the methods comprise (e.g., for each subject, if more than one subject is implicated) monitoring the subject for TLS by performing a serum potassium assay on the subject at the end of the alvocidib administration and about two hours after the end of the alvocidib administration, and performing a laboratory TLS panel on the subject about four hours after the end of the alvocidib administration; and administering to the subject an effective amount of one or more TLS therapies if the subject has an elevated serum potassium level or an abnormal laboratory TLS panel. In another aspect, the methods comprise (e.g., for each subject, if more than one subject is implicated) monitoring the subject for TLS by performing a laboratory TLS panel on the subject about three to about four hours after the end of the alvocidib administration; and administering to the subject an effective amount of one or more TLS therapies if the subject has an abnormal laboratory TLS panel. It will be appreciated that when more than one subject is implicated by a particular preamble, the recited monitoring and administering steps should be carried out with respect to each subject individually.

“Reducing the severity of TLS,” as used herein, includes improving a condition, symptom, disorder, or parameter associated with TLS, e.g., to a clinically meaningful extent. Reducing the severity of TLS can be evidenced by reducing TLS from grade 5 to grade 3 or 4, from grade 4 or 5 to grade 3, and/or eliminating grade 3, 4 or 5 TLS altogether, according to the CTCAE 5.0 TLS grading scale. Reducing the severity of TLS can also be evidenced by a reduction in the severity of a symptom associated with TLS.

“Reducing the incidence of TLS,” as used herein, includes preventing TLS (e.g., grade 3, grade 4 and/or grade 5 TLS, according to CTCAE 5.0 TLS grading), e.g., the occurrence or re-occurrence of TLS, in an individual subject, and reducing the occurrence, rate or frequency of TLS in a population of subjects.

“Decreasing mortality from TLS,” as used herein, includes preventing death from TLS, or a sequela thereof, in an individual subject, and reducing the occurrence, rate or frequency of death from TLS, or a sequela thereof, in a population of subjects. It is understood that a death may not always be solely or definitively attributable to particular cause(s), particularly not in the context of cancer treatment. “From TLS” thus includes mortalities for which TLS, or a sequela thereof, is a substantial and/or likely contributing factor in addition to mortalities for which TLS, or a sequela thereof, is the sole and/or definitive cause of the mortality. Methods and criteria for attributing a death to a particular cause(s) are known in the art. Typically, a case is evaluated for factors such as concomitant medication toxicity, co-morbidities (e.g., cardiac history, infection), and underlying cancer diagnosis/symptoms, as well as known toxicities of a drug and the temporal relationship between when the drug was administered and the onset of symptoms in making this determination.

“Increasing survival time,” as used herein, includes prolonging the life of an individual subject, e.g., beyond the mean and/or median survival time associated with a particular cancer and/or therapeutic regimen, and extending the mean and/or median survival time of a population of subjects, e.g., beyond the mean and/or median survival time associated with a particular cancer and/or therapeutic regimen.

While reducing the severity of TLS in, reducing the incidence of TLS in, monitoring for the development of TLS while treating, decreasing mortality from TLS in, increasing survival time of, treating TLS in, preventing TLS in, and/or diagnosing and/or treating TLS in a hematological cancer subject being treated with alvocidib can be done at the level of an individual subject, such methods can also be applied to populations of subjects, and assessed at a population-wide level, for example, as is commonly done in the context of a clinical trial. Assessment of these methods at the individual and the population-wide level, including selection of and comparison to appropriate controls and/or comparators, is within the abilities of a person of ordinary skill in the relevant art.

TLS therapies are administered in accordance with good clinical practices. In one embodiment, when a subject has a serum potassium level greater than 4.0 mEq/L, at least one of the TLS therapies comprises administering to the subject about 30 g (e.g., 30 g) of sodium polystyrene sulfonate. In another embodiment, when the subject has a serum potassium level greater than 5.0 mEq/L, at least one of the TLS therapies comprises administering to the subject about 10 units (e.g., 10 units) of IV rapid-acting insulin and about 25 g (e.g., 25 g) of IV dextrose 50%. In a further embodiment, when a subject has a serum potassium level greater than 5.5 mEq/L, the TLS therapy further comprises emergent, intermittent or continuous dialysis.

In some of the embodiments, the methods in accordance with the present disclosure include performing additional diagnostic assays such as a lactate dehydrogenase assay at least once every 24 hours after the end of alvocidib administration. In another embodiment, the methods further comprise performing a fibrinogen assay on a subject prior to the start of the administration of alvocidib. Additional assays for fibrinogen may optionally be performed at a frequency as determined by the skilled artisan.

In addition to monitoring for the development of TLS, the methods of the present disclosure can include one or more prophylactic or other pre-treatments. In one embodiment, the methods further comprise administering to the subject (e.g., a prophylactically effective amount of) intravenous (IV) hydration (e.g., at a rate of from about 50 to about 750 cc/hour, from about 100 to about 500 cc/hour, from about 250 to about 500 cc/hour, about 100 cc/hour, 200 cc/hour, 250 cc/hour, 300 cc/hour, 350 cc/hour, 400 cc/hour, 450 cc/hour or 500 cc/hour), e.g., beginning about 24 hours, about 12 hours, about 6 hours, about 2 hours or about 1 hour prior to the administration of alvocidib. In one embodiment, the methods further comprise administering to the subject continuous IV hydration. In one embodiment, the methods further comprise administering to the subject continuous IV hydration at a rate of about 100 cc/hr. As used herein, the term “IV hydration” means 0.45% NaCl aqueous solution, or similar hydration fluid.

In some embodiments, administration of continuous IV hydration can begin about 10 hours prior to the start of the administration of alvocidib. Administration of continuous IV hydration can continue for at least 24 hours after the end of the alvocidib administration.

In some embodiments, IV hydration is administered prior to the start of the alvocidib administration (e.g., during the time period leading up to the alvocidib administration, such that, for example, alvocidib administration commences immediately or almost immediately (within 30 minutes) of completion of IV hydration). In some embodiments, IV hydration is administered beginning at least two hours (e.g., about one to about two hours, about one hour, about two hours) prior to the start of the alvocidib administration, and continues for at least two hours (e.g., the at least two hours, from about one hour to about two hours, about one hour, about two hours). In some embodiments, IV hydration is also or alternatively administered for from about one to about two hours, beginning at the end of the alvocidib administration.

In certain embodiments, the subject is also administered an effective amount (e.g., a prophylactically effective amount) of allopurinol (e.g., from about 300 mg to about 600 mg per day) and/or an effective amount (e.g., a prophylactically effective amount) of an oral phosphate binder. Concurrent with the administration of continuous IV hydration, in certain embodiments, the subject is also administered an effective amount of allopurinol, at the start of the administration of IV hydration and, optionally, an effective amount of an oral phosphate binder (e.g., at the start of the administration of IV hydration). In certain embodiments, the subject is administered an effective amount of allopurinol, beginning at the start of the administration of IV hydration and/or an effective amount of an oral phosphate binder, beginning at the start of the administration of IV hydration. In certain embodiments, the subject is administered an effective amount of allopurinol beginning at least or about 72 hours, at least or about 48 hours, at least or about 36 hours, at least or about 24 hours, at least or about 12 hours, at least or about 6 hours prior to the start of the alvocidib administration, or at the start of the alvocidib administration and/or an effective amount of an oral phosphate binder, beginning at the start of the administration of continuous IV hydration. Typically, administration of allopurinol and/or the oral phosphate binder will continue throughout the first cycle of treatment (e.g., to day 28 of a 28-day treatment cycle), although in some embodiments, administration of allopurinol and/or an oral phosphate binder will independently continue for one, two, three, four, five, six, seven, eight, nine, 10, 11, 12, 13, 14 or 15 days, for the first cycle of treatment, or for each day of dosing, e.g., of alvocidib of the alvocidib-containing treatment regimen. In some embodiments, administration of allopurinol continues throughout the first cycle of treatment, and administration of the oral phosphate binder continues for seven days. Effective amounts (e.g., therapeutically effective amounts, prophylactically effective amounts) of allopurinol and/or an oral phosphate binder are well-known in the art, and/or can be readily determined by a skilled artisan.

Examples of oral phosphate binders include, but are not limited to, calcium acetate, sevelamer, ferric citrate, lanthanum carbonate, sucroferric oxyhydroxide and aluminum hydroxide.

In certain embodiments, the subject is administered an effective amount (e.g., a prophylactically effective amount, a therapeutically effective amount) of rasburicase. For example, a prophylactically effective amount of rasburicase can be administered to high-risk subjects. Effective amounts (e.g., therapeutically effective amounts, prophylactically effective amounts) of rasburicase are well-known in the art, and/or can be readily determined by a skilled artisan.

In certain embodiments, the methods comprise administering a prophylactically effective amount of each of: IV hydration, allopurinol and an oral phosphate binder to the subject; monitoring the subject for TLS by performing a laboratory TLS panel on the subject about three to about four hours after the end of the alvocidib administration; and administering to the subject a therapeutically effective amount of one or more TLS therapies if the subject has an abnormal laboratory TLS panel. In some embodiments, the methods further comprise administering a therapeutically effective amount of alvocidib to the subject.

In other embodiments, the methods comprise administering a prophylactically effective amount of each of: IV hydration (e.g., continuous IV hydration), allopurinol and an oral phosphate binder to the subject; monitoring the subject for TLS by performing a serum potassium assay on the subject at the end of the alvocidib administration and about two hours after the end of the alvocidib administration, and performing a laboratory TLS panel on the subject about four hours after the end of the alvocidib administration; and administering to the subject a therapeutically effective amount of one or more TLS therapies if the subject has an elevated serum potassium level or an abnormal laboratory TLS panel. In some embodiments, the methods further comprise administering a therapeutically effective amount of alvocidib to the subject.

### EXAMPLES

### Example 1. Alvocidib, Cytarabine, Mitoxantrone (ACM) Treatment for Patients at High Risk and Not at High Risk for TLS

Patients receive treatment with ACM over days 1-9 as follows: On days 1, 2 and 3, alvocidib (A) is administered 30 mg/m² as a 30-minute intravenous (IV) bolus followed by 60 mg/m² over 4 hours as an IV infusion. Days 4 and 5 are rest days with no chemotherapy treatment given. On days 6, 7 and 8, cytarabine (C) is administered continuously over 72 hours. Dosing of cytarabine is 2 gm/m² by continuous IV infusion over 72 hours (i.e., 667 mg/m² daily, for a total of 2 gm/m²). On day 9, mitoxantrone hydrochloride (M) is administered 12 hours after completion of cytarabine treatment at 40 mg/m² by IV infusion over 1-2 hours.

Management of Hyperkalemia and Tumor Lysis Syndrome for Patients Receiving ACM Treatment

Laboratory indicators of tumor lysis syndrome (TLS) are monitored in addition to the therapies listed below. Tumor lysis laboratory evaluations include electrolytes (sodium, potassium, chloride, and carbon dioxide), as well as creatinine, calcium, lactate dehydrogenase (LDH), uric acid, and phosphorus levels.

Monitoring Patients at High Risk for TLS

For patients at high risk for TLS, including those with risk scores of ≥2 according to Table 1, those with monocytic leukemia phenotype, or those with a history of FLT3 positive AML, the patient is monitored as follows: a STAT, or otherwise rapid result, serum potassium is obtained at the end of the alvocidib infusion on day 1. About 2 hours after the end of the alvocidib infusion a STAT, or otherwise rapid result, serum potassium is obtained. A full laboratory TLS panel is obtained at about 4 hours after the end of the alvocidib infusion.

If evidence of clinically meaningful TLS is observed in any of the first serum potassium, second serum potassium or the laboratory TLS panel, a laboratory TLS panel is obtained about every 2 hours during the first 24 hours after the end of the alvocidib infusion. (LDH levels are optionally recommended to be assessed at least once every 24 hours.) If no evidence of clinically meaningful TLS is observed in any of the first serum potassium, second serum potassium or the third laboratory TLS panel, a laboratory TLS panel is obtained about every 4 hours during the first 24 hours after the end of the alvocidib infusion. (LDH levels are optionally recommended to be assessed at least once every 24 hours.)

If, after the first 24 hours after the end of the alvocidib infusion, there is no evidence of clinically meaningful TLS, a laboratory TLS panel is obtained about every 6 hours for the remainder of alvocidib treatment and then about every 12 hours after completion of alvocidib treatment during the cytarabine and mitoxantrone treatments. (LDH levels are optionally recommended to be assessed at least once every 24 hours.)

Additionally, fibrinogen levels are monitored at baseline and then as clinically indicated. Patients who are determined to be at intermediate- or high-risk for TLS should be considered for rasburicase prophylaxis according to institutional standards.

Monitoring Patients not at High Risk for TLS

During the first 24 hours, laboratory TLS panels are monitored at the start of the alvocidib treatment and about every 4 hours thereafter. Laboratory studies during this period should be run as a “STAT” to ensure the results are available in a timely manner. (LDH levels are optionally recommended to be assessed at least once every 24 hours.) If there is no evidence of TLS during the first 24 hours, then tumor lysis labs are monitored about every 6 hours for the remainder of the alvocidib treatment, and then about every 12 hours after the completion of the alvocidib treatment during the cytarabine and mitoxantrone treatments. (LDH levels are recommended to be assessed at least once every 24 hours.) Additionally, fibrinogen levels are monitored at baseline and then as clinically indicated.

Treatment for all Patients Receiving ACM

In addition to the monitoring protocol above, patients receive IV hydration with 0.45% NaCl (or similar hydration fluid per institutional standard) sterile solution at 100 cc/hour for at least 10 hours prior to the first dose of chemotherapy being administered (optional for subsequent cycles). If, by Day 4, there is no evidence of tumor lysis syndrome, the hydration rate can be reduced to a maintenance level.

Urine output is diligently monitored to ensure that fluid output equals fluid input. If input is greater than output by 10%, administration of diuretics is encouraged. Replacement of excessive fluid losses, including from diarrhea, should be done unless otherwise clinically indicated.

Allopurinol is administered orally each day of dosing (e.g., for the first cycle) to be started at the same time as the initiation of IV hydration.

Oral phosphate binder is to be started at the same time as initiation of IV hydration, unless contraindicated.

Evaluation after Cycle 1

Patients who achieve CR, CRi, or PR after the first cycle (completion of all doses) may receive up to 3 additional optional cycles of treatment.

After completing the first cycle of treatment, continued use of mitoxantrone is optional. Mitoxantrone must be omitted from subsequent cycles if the patient's lifetime daunorubicin equivalent exceeds 460 mg/m² or the left ventricular ejection fraction (LVEF) drops below 45%.

Patients not demonstrating evidence of CR, CRi, or PR after the first cycle of treatment will be considered for removal from the study, although with permission of the Medical Monitor, induction treatment may continue if clinically indicated and provided there is no evidence of toxicity ≥NCI CTCAE grade 4.

### Example 2. Treatment for Patients Having Newly Diagnosed AML

This study will evaluate the safety and efficacy of alvocidib in combination with cytarabine/daunorubicin (7+3) in patients with newly diagnosed AML. Treatment consists of increasing dose levels of alvocidib starting at 20 mg/m² as a 30-minute IV bolus followed by 30 mg/m² over 4 hours on days 1-3, cytarabine 100 mg/m²/day by continuous IV infusion on days 5-11, followed by (e.g., followed about 30 minutes later by) daunorubicin 60 mg/m² IV on days 5-7. Reinduction therapy with alvocidib (same dose as induction) days 1-3, followed by cytarabine 100 mg/m²/day continuous IV on days 5-9, and daunorubicin 45 mg/m² IV days 5-6 is recommended in patients with >10% and >5% cellularity and blasts, respectively.

TLS Prevention and Treatment

Mandatory IV hydration with 0.45% NaCl (or similar hydration fluid per institutional standard) sterile solution at 100 cc/hour for at least 10 hours prior to initiation of the first dose of chemotherapy during induction (optional for subsequent cycles, including reinduction) will be provided. If, by Day 4, there is no evidence of TLS, the hydration rate can be reduced to a maintenance level.

Diligent monitoring of urine output should be done to ensure that fluid output equals fluid input. If input is greater than output by 10%, administration of diuretics is encouraged. Replacement of excessive fluid losses, including from diarrhea, should be done unless otherwise clinically indicated.

Mandatory allopurinol will be given orally each day of dosing for the first cycle (optional in subsequent cycles), and will be started at the same time IV hydration is initiated. Mandatory oral phosphate binder will be started at the same time as IV hydration is initiated (optional in subsequent cycles), unless contraindicated.

Laboratory indicators of TLS will be monitored during induction therapy. Evaluation of laboratory indicators of TLS may be adjusted for reinduction and consolidation therapies based on the extent of the tumor burden. Monitoring of laboratory indicators of TLS will include obtaining tumor lysis laboratories, including electrolytes (sodium, potassium, chloride, and carbon dioxide), as well as creatinine, calcium, lactate dehydrogenase (LDH), uric acid, and phosphorus levels.

For patients at high risk for TLS, including those with risk scores of greater than or equal to 2 according to Table 1, those with monocytic leukemia phenotype, or those with a history of FLT3 positive AML, monitoring will include: (i) obtaining a STAT serum potassium at the end of an alvocidib infusion; (ii) obtaining a serum potassium 2 hours after the end of an alvocidib infusion, (iii) obtaining a full TLS panel 4 hours after the end of an alvocidib infusion, and (iv) monitoring fibrinogen levels at baseline and then as clinically indicated. If there is evidence of clinically meaningful TLS, a TLS panel will be obtained every 2 hours during the first 24 hours after the end of an alvocidib infusion (assessment of LDH levels at least once every 24 hours is also recommended). If there is no evidence of clinically meaningful TLS, a TLS panel will be obtained every 4 hours during the first 24 hours after the end of an alvocidib infusion (assessment of LDH levels at least once every 24 hours is also recommended). If, after the first 24 hours after the end of an alvocidib infusion, there is no evidence of TLS, a TLS panel will be obtained approximately every 6 hours for the remainder of alvocidib treatment, and then every 12 hours after completion of alvocidib during daunorubicin and cytarabine treatment (assessment of LDH levels at least once every 24 hours is also recommended).

In addition, patients who are determined to be at intermediate- or high-risk for TLS should be considered for rasburicase prophylaxis according to institutional standards.

For patients not at high risk for TLS, tumor lysis evaluations will be monitored at the start of alvocidib treatment and approximately every 4 hours thereafter for 24 hours. Laboratory studies during this period will be run as a “STAT” to ensure the results are available in a timely manner. If there is no evidence of TLS during first the 24 hours, then tumor lysis evaluations will be monitored approximately every 6-8 hours for the remainder of alvocidib treatment, and then every 12 hours after the completion of alvocidib during cytarabine and daunorubicin treatment. Fibrinogen levels will be monitored at baseline and then as clinically indicated.

Guidelines for TLS Management

If potassium levels are increasing to greater than 4.0 mEq/L, patients should receive a 30 g dose of sodium polystyrene sulfonate, unless there are other likely causes of hyperkalemia other than TLS or contraindication to its use. If potassium levels rise to greater than 5.0 mEq/L, in addition to the 30 g dose of sodium polystyrene sulfonate, patients should also receive 10 units of IV rapid-acting insulin and 25 gm (1 ampoule) of IV dextrose 50%, unless there are other likely causes of hyperkalemia other than TLS or contraindication to its use. If potassium levels rise to greater than 5.5 mEq/L, patients should be considered for emergent, intermittent or continuous dialysis.

Calcium supplementation should only be given for symptomatic hypocalcemia in this setting to avoid renal precipitation of calcium phosphate crystals.

Patients who develop clinical evidence of cytokine release syndrome or who have hyperkalemia requiring dialysis will receive immediate steroid therapy with an equivalent of at least 20 mg of IV dexamethasone.

At the first signs of diarrhea, patients should initiate loperamide (or equivalent) 2 mg by mouth every 2 hours during the waking hours. Once the diarrhea is controlled, the time interval of loperamide may be titrated to a frequency that adequately controls the diarrhea. The diarrhea observed with alvocidib almost always resolves following completion of therapy, so treatment with loperamide following completion of therapy will not be required in most patients. If loperamide (or equivalent) does not control diarrhea, cholestyramine (or equivalent) 5 g orally 4 times daily may be added. For patients developing diarrhea during alvocidib administration, subsequent treatments should include a similar diarrhea prophylaxis. If diarrhea is not controlled with the above prophylactic regimen and is Grade 2 or greater, therapy should be held until diarrhea has resolved. Replacement of excessive fluid losses should be done unless otherwise clinically indicated.

### Example 3. Treatment for Patients Having Relapsed/Refractory AML Following Treatment with Venetoclax Combination Therapies

This study will evaluate the safety and efficacy of alvocidib in patients with AML who have either relapsed from (i.e., experience reoccurrence of disease following a CR/CRᵢ with duration of greater than or equal to 90 days) or are refractory to (i.e., failed to achieve a CR/CRᵢ, or achieved a CR/CRᵢ with duration of less than 90 days) venetoclax in combination with azacytidine or decitabine.

Stage 1 of the study is randomized and consists of two arms (26 patients per arm). Those patients in Arm 1 are given alvocidib and low dose cytarabine (LDAC) on a 28-day treatment cycle. On Day 1, patients in Arm 1 are given 25 mg/m² alvocidib as a 30-60-minute intravenous (IV) bolus. On Days 3 through 12 (10 days), patients in Arm 1 are given 20 mg/m² cytarabine by subcutaneous (SC) injection each day. On Day 15, patients in Arm 1 are given 50 mg/m² alvocidib as a 30-60-minute IV bolus. Those patients in Arm 2 of the study are given alvocidib on a 28-day treatment cycle. On Day 1, patients in Arm 2 are given 25 mg/m² alvocidib as a 30-60-minute IV bolus. On Days 8 and 15, patients in Arm 2 are given 50 mg/m² alvocidib as a 30-60-minute IV bolus.

Stage 2 of the study consists of 76 patients, who will be dosed with a regimen selected based on Stage 1 performance.

### TLS Prevention and Treatment in an Outpatient Setting

Tumor lysis may occur as part of initial cytoreductive therapy. The most extreme form, known as TLS, is characterized by hyperkalemia, hyperuricemia, hyperphosphatemia, increased lactate dehydrogenase (LDH), coagulopathy, and a potential cytokine release syndrome.

To prevent and/or treat TLS, mandatory IV hydration with 0.45% NaCl (or similar hydration fluid per institutional standard) sterile solution at 500 cc for 1-2 hours or at least 2 hours prior to alvocidib will be provided, then an additional 500 cc for 1-2 hours after alvocidib during Cycle 1 (optional for subsequent cycles). Replacement of excessive fluid losses, including from diarrhea, should be done unless otherwise clinically indicated, as alvocidib is known to induce mild diarrhea during treatment days. Over-the-counter measures are typically effective in this setting if initiated early. Persistent diarrhea despite optimal outpatient management would trigger medical consultation.

Mandatory oral allopurinol will be started at least 72 hours prior to Day 1 of Cycle 1 and continued until completion of the first cycle (e.g., 28 days). Oral allopurinol may be discontinued for subsequent treatment cycles if uric acid levels are within normal limits and there is no evidence of TLS.

Mandatory oral phosphate binder will be started at the same time as initiation of IV hydration on Day 1 of Cycle 1 and continued for the first week. If serum phosphorus levels are <3 after the first treatment with alvocidib and there is no evidence of TLS, phosphate binders may be discontinued. Patients should continue to be monitored for TLS as outlined for subsequent treatment cycles. Caution is warranted for patients who still have a high blast count as they remain at risk for TLS with subsequent treatments.

Laboratory indicators of TLS will be monitored during Cycle 1 according to the following protocol: (i) tumor lysis laboratory evaluations, including electrolytes (sodium, potassium, chloride, and carbon dioxide), as well as creatinine, calcium, lactate dehydrogenase, uric acid, and phosphorous levels will be obtained prior to alvocidib infusion and 2 hours±30 minutes following completion of IV hydration post alvocidib administration, as well as daily for the first three days following the first alvocidib treatment (Days 2-4), and at least weekly for the remainder of Cycle 1, and (ii) fibrinogen levels will be monitored at baseline and as clinically indicated.

During subsequent cycles, tumor lysis laboratory evaluations will be assessed prior to each dose of alvocidib.

Guidelines for TLS Management

If potassium levels are increasing to greater than 4.0 mEq/L, patients should receive a 30 g dose of sodium polystyrene sulfonate, unless there are other likely causes of hyperkalemia other than TLS or contraindication to its use. If potassium levels rise to greater than 5.0 mEq/L, in addition to the 30 g dose of sodium polystyrene sulfonate, patients should also receive 10 units of IV rapid-acting insulin and 25 gm (1 ampoule) of IV dextrose 50%, unless there are other likely causes of hyperkalemia other than TLS or contraindication to its use. Investigators are strongly encouraged to consider patient hospitalization for inpatient monitoring and follow up. If potassium levels rise to greater than 5.5 mEq/L, patients should be considered for emergent, intermittent or continuous dialysis.

Calcium supplementation should only be given for symptomatic hypocalcemia in this setting to avoid renal precipitation of calcium phosphate crystals.

Patients who develop clinical evidence of cytokine release syndrome or who have hyperkalemia requiring dialysis will receive immediate steroid therapy with an equivalent of at least 20 mg of IV dexamethasone.

At the first signs of diarrhea, patients should initiate loperamide (or equivalent) 2 mg by mouth every 2 hours during the waking hours. Once the diarrhea is controlled, the time interval of loperamide may be titrated to a frequency that adequately controls the diarrhea. The diarrhea observed with alvocidib almost always resolves following completion of therapy, so treatment with loperamide following completion of therapy will not be required in most patients. If loperamide (or equivalent) does not control diarrhea, cholestyramine (or equivalent) 5 g orally 4 times daily may be added. If diarrhea is not controlled with the above prophylactic regimen and is Grade 2 or greater, patients should contact the clinic and study drug treatment should be held until diarrhea has resolved. Should diarrhea persist beyond Cycle 1, patients should undergo testing for Clostridioides difficile (C. difficile). Should testing indicate the presence of C. difficile, appropriate antibiotics targeting this infection should be initiated. Should testing exclude the presence of C. difficile, diarrhea prophylaxis similar to Cycle 1 should be continued in subsequent cycles. Replacement of excessive fluid losses should be done unless otherwise clinically indicated.

### Example 4. ACM Treatment for Patients at High Risk and not at High Risk for TLS

Patients were treated with ACM according to the treatment regimen described in Example 1, and monitored for TLS according to the monitoring protocols described in Example 1. Briefly, patients with relapsed or refractory AML or newly-diagnosed, high-risk AML received treatment with ACM over days 1-9 as follows: On days 1, 2 and 3, alvocidib (A) was administered 30 mg/m² as a 30-minute intravenous (IV) bolus followed by 60 mg/m² over 4 hours as an IV infusion. Days 4 and 5 were rest days with no chemotherapy treatment given. On days 6, 7 and 8, cytarabine (C) was administered continuously over 72 hours. Dosing of cytarabine was 2 gm/m² by continuous IV infusion over 72 hours (i.e., 667 mg/m² daily, for a total of 2 gm/m²). On day 9, mitoxantrone hydrochloride (M) was administered 12 hours after completion of cytarabine treatment at 40 mg/m² by IV infusion over 1-2 hours.

In this study, patients were prescreened to determine percent MCL-1 dependence, and placed into the following groups. One group consisted of patients with AML with demonstrated MCL-1 dependence of ≥40% by mitochondrial profiling in bone marrow who are either in first relapse (within 24 months of CR) or have primary refractory AML (i.e., no CR or CRi after 2 cycles of intensive anthracycline/cytarabine±etoposide or cladribine induction). A second group consisted of patients with newly-diagnosed, high-risk AML, as demonstrated by MCL-1 dependence of ≥40% by mitochondrial profiling in bone marrow. Patients in first relapse (within 24 months of CR) or having primary refractory AML (i.e., no CR or CRi after two cycles of intensive anthracycline/cytarabine±etoposide or cladribine induction) with lesser MCL-1 dependencies were allocated into one of the following three groups: patients with demonstrated MCL-1 dependence of 30-<40% were allocated into Arm A, patients with demonstrated MCL-1 dependence of 15%-<30% were allocated into Arm B, and patients with demonstrated MCL-1 dependence of 0-<15% were allocated into Arm C, where MCL-1 dependence was determined by mitochondrial profiling in bone marrow.

In addition, the following guidelines were recommended for management of high risk patients: If potassium levels are increasing to greater than 4.0 mEq/L, patients should receive a 30 g dose of sodium polystyrene sulfonate, unless there are other likely causes of hyperkalemia other than TLS or contraindication to its use. If potassium levels rise to greater than 5.0 mEq/L, in addition to the 30 g dose of sodium polystyrene sulfonate, patients should also receive 10 units of IV rapid-acting insulin and 25 gm (1 ampoule) of IV dextrose 50%, unless there are other likely causes of hyperkalemia other than TLS or contraindication to its use. If potassium levels rise to greater than 5.5 mEq/L, patients should be considered for emergent, intermittent or continuous dialysis.

The following guidelines were also recommended: Calcium supplementation should only be given for symptomatic hypocalcemia in this setting to avoid renal precipitation of calcium phosphate crystals. Patients who develop clinical evidence of cytokine release syndrome or who have hyperkalemia requiring dialysis will receive immediate steroid therapy with an equivalent of at least 20 mg of IV dexamethasone. At the first signs of diarrhea, patients should initiate loperamide (or equivalent) 2 mg by mouth every 2 hours during the waking hours. Once the diarrhea is controlled, the time interval of loperamide may be titrated to a frequency that adequately controls the diarrhea. The diarrhea observed with alvocidib almost always resolves following completion of therapy, so treatment with loperamide following completion of therapy will not be required in most patients. If loperamide (or equivalent) does not control diarrhea, cholestyramine (or equivalent) 5 g orally 4 times daily may be added. For patients developing diarrhea during alvocidib administration, subsequent treatments should include a similar diarrhea prophylaxis. If diarrhea is not controlled with the above prophylactic regimen and is Grade 2 or greater, therapy should be held until diarrhea has resolved. Replacement of excessive fluid losses should be done unless otherwise clinically indicated.

The number of treatment-emergent adverse events classified as TLS for various safety populations are reported in Table 2.

TABLE 2
Treatment-emergent Adverse Events by Safety Population

| | ACM | ACM | ACM | | |
|---|---|---|---|---|---|
| | Relapsed/ | Relapsed/ | Relapsed/ | ACM/CM | ACM |
| | Refractory | Refractory | Refractory | Relapsed/ | Newly |
| | 0 to <15% | 15 to <30% | 30 to <40% | Refractory ≥40% | Diagnosed ≥40% |
| | MCL-1 | MCL-1 | MCL-1 | MCL-1 | MCL-1 |
| | Exploratory | Exploratory | Exploratory | Stage 1 & 2 | Exploratory |
| | (N = 15) | (N = 14) | (N = 14) | (N = 42) | (N = 14) |
| Tumor Lysis | 4 (26.7%) | 1 (7.1%) | 3 (21.4%) | 8 (19.0%) | 3 (21.4%) |
| Syndrome | | | | | |

In this study, there were 17 cases of grade 3 TLS, and two cases of grade 4 TLS. In the grade 3 group, three patients had doses interrupted, and one had a dose delay due to TLS, and in the grade 4 group, only one patient had a dose interrupted. There were no instances of grade 5 TLS in this study.

### Example 5. Treatment for Patients Having Newly Diagnosed AML

Patients were treated with ACD according to a treatment regimen similar to that described in Example 2, and monitored for TLS according to the monitoring protocols described in Example 2. Briefly, treatment consisted of increasing dose levels of alvocidib starting at 20 mg/m² as a 30-minute (±10 minutes) IV bolus followed by 30 mg/m² as a 4-hour (±15 minutes) IV infusion on days 1-3, followed by daunorubicin 60 mg/m² IV on days 5-7 via IV bolus of up to 15 minutes (±5 minutes) in duration each day, followed by (e.g., followed immediately by) cytarabine 100 mg/m²/day by 24-hour (±2 hours) continuous IV infusion on days 5-11. Reinduction therapy with alvocidib (same dose as induction) days 1-3, followed by daunorubicin 45 mg/m² IV via IV bolus of up to 15 minutes (±5 minutes) in duration each day on days 5-6, followed by (e.g., followed immediately by) cytarabine 100 mg/m²/day continuous, 24-hour IV infusion (±2 hours) on days 5-9, was recommended in patients with evidence of residual leukemia, defined as >5% bone marrow blasts plus >10% cellularity, based on day 14 (±3 days) bone marrow.

Dose levels of alvocidib tested included 10 mg/m² as a 30-minute (±10 minutes) IV bolus followed by 15 mg/m² as a 4-hour (±15 minutes) IV; 20 mg/m² as a 30-minute (±10 minutes) IV bolus followed by 30 mg/m² as a 4-hour (±15 minutes) IV; 30 mg/m² as a 30-minute (±10 minutes) IV bolus followed by 40 mg/m² as a 4-hour (±15 minutes) IV; 30 mg/m² as a 30-minute (±10 minutes) IV bolus followed by 50 mg/m² as a 4-hour (±15 minutes) IV; and 30 mg/m² as a 30-minute (±10 minutes) IV bolus followed by 60 mg/m² as a 4-hour (±15 minutes) IV.

The number of treatment-emergent adverse events classified as TLS for various safety populations are reported in Table 3.

TABLE 3
Treatment-emergent Adverse Events by Safety Population

| | Alvocidib | Alvocidib | Alvocidib | Alvocidib | |
|---|---|---|---|---|---|
| | 20/30 | 30/40 | 30/50 | 30/60 | Study |
| | mg/m² | mg/m² | mg/m² | mg/m² | Total |
| | (N = 3) | (N = 3) | (N = 3) | (N = 22) | (N = 31) |
| Tumor lysis | 0 | 1 (33.3%) | 0 | 7 (31.8%) | 8 (25.8%) |
| syndrome | | | | | |

In this study, there were seven cases of grade 3 TLS, and one case of grade 4 TLS. None were considered a dose-limiting toxicity, and only one (the grade 4 case) delayed the dosing of the patient. There were no instances of grade 5 TLS in this study.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A method of treating a hematological cancer in a subject comprising:
A. administering an effective amount of alvocidib to the subject;
B. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
C. administering to the subject an effective amount of one or more TLS therapies if the subject has an elevated serum potassium level or an abnormal laboratory TLS panel.

2. The method of claim 1, wherein the monitoring further comprises:
iii. performing an additional laboratory TLS panel on the subject about every two hours after the laboratory TLS panel in (ii), until twenty-four hours after the end of the alvocidib administration.

3. The method of claim 1, wherein the monitoring further comprises:
iii. performing an additional laboratory TLS panel on the subject about every four hours after the laboratory TLS panel in (ii), until twenty-four hours after the end of the alvocidib administration.

4. The method of claim 1 or 2, wherein the subject exhibits evidence of clinically meaningful TLS after the alvocidib administration.

5. The method of claim 1 or 3, wherein the subject does not exhibit evidence of clinically meaningful TLS after the alvocidib administration.

6. The method of any one of claims 1 to 5, wherein an elevated serum potassium level is detected in at least one of the serum potassium assay or the laboratory TLS panel.

7. The method of claim 6, wherein the elevated serum potassium level is selected from greater than 4.0 mEq/L, greater than 5.0 mEq/L, greater than 5.5 mEq/L, or a level that is at least 25% above the subject's baseline level.

8. The method of claim 7, wherein the elevated serum potassium level is greater than 4.0 mEq/L, and the one or more TLS therapies comprise administering to the subject about 30 g of sodium polystyrene sulfonate.

9. The method of claim 8, wherein the elevated serum potassium level is greater than 5.0 mEq/L, and the one or more TLS therapies further comprise administering to the subject about 10 units of intravenous (IV) rapid-acting insulin and about 25 g of IV dextrose 50%.

10. The method of claim 9, wherein the elevated serum potassium level is greater than 5.5 mEq/L, and the one or more TLS therapies further comprise emergent, intermittent or continuous dialysis.

11. The method of any one of claims 1 to 10, wherein the method further comprises performing a lactic acid dehydrogenase (LDH) assay on the subject at least once every 24 hours after the end of alvocidib administration.

12. The method of any one of claims 1 to 11, wherein the method further comprises administering to the subject continuous intravenous (IV) hydration.

13. The method of claim 12, wherein the continuous IV hydration administration begins about 10 hours prior to the start of the alvocidib administration.

14. The method of claim 12 or 13, wherein the continuous IV hydration is administered for at least 24 hours after the end of the alvocidib administration.

15. The method of any one of claims 12 to 14, wherein the method further comprises administering to the subject an effective amount of allopurinol, beginning at the start of the administration of IV hydration.

16. The method of any one of claims 12 to 15, wherein the method further comprises administering to the subject an effective amount of an oral phosphate binder, beginning at the start of the administration of IV hydration.

17. The method of any one of claims 1 to 16, wherein the method further comprises performing a fibrinogen assay on the subject prior to the start of the alvocidib administration.

18. The method of any one of claims 1 to 17, wherein the effective amount of alvocidib is administered to the subject as one or more hybrid doses.

19. The method of any one of claims 1 to 18, wherein the effective amount of alvocidib is administered to the subject as a 30-minute IV bolus of alvocidib followed by a 4-hour IV infusion of alvocidib.

20. The method of claim 19, wherein the 30-minute IV bolus is 30 mg/m² of alvocidib.

21. The method of claim 19 or 20, wherein the 4-hour IV infusion is 60 mg/m² of alvocidib.

22. The method of any one of claims 1 to 21, wherein the effective amount of alvocidib is administered to the subject once a day for at least one day.

23. The method of claim 22, wherein the effective amount of alvocidib is administered to the subject once a day for at least two days.

24. The method of claim 23, wherein the effective amount of alvocidib is administered to the subject once a day for three days.

25. The method of any one of claims 1 to 24, wherein the method further comprises administering to the subject an effective amount of at least one cytotoxic drug after the alvocidib administration.

26. The method of claim 25, wherein the at least one cytotoxic drug is cytarabine.

27. The method of claim 25 or 26, wherein the subject is administered an effective amount of at least two cytotoxic drugs after the alvocidib administration.

28. The method of claim 27, wherein the at least two cytotoxic drugs are cytarabine and mitoxantrone.

29. The method of claim 26 or 28, wherein the cytarabine is administered 5 days after the alvocidib is first administered.

30. The method of claim 26, 28 or 29, wherein the cytarabine is administered as a continuous IV infusion for about 72 hours.

31. The method of any one of claims 26 and 28 to 30, wherein the effective amount of cytarabine is 667 mg/m² per 24 hours, for a total of 2 gm/m².

32. The method of any one of claims 28 to 31, wherein the mitoxantrone is administered about 12 hours after the end of the cytarabine administration.

33. The method of any one of claims 28 to 32, wherein the mitoxantrone is administered as an intravenous (IV) infusion over about 1 to about 2 hours.

34. The method of any one of claims 28 to 33, wherein the effective amount of mitoxantrone is 40 mg/m².

35. The method of claim 27, wherein the at least two cytotoxic drugs are daunorubicin or idarubicin, and cytarabine.

36. The method of any one of claims 1 to 35, wherein the method further comprises obtaining a biological sample from the subject prior to the alvocidib administration, and determining a MCL-1 dependence score from the sample.

37. The method of claim 36, wherein the MCL-1 dependence score is selected from greater than or equal to 40%, 30% to less than 40%, 15% to less than 30%, or 0 to less than 15%.

38. The method of any one of claims 1 to 37, wherein the method further comprises determining whether the subject is a high risk TLS subject prior to the alvocidib administration.

39. The method of any one of claims 1 to 38, wherein the subject is a high risk TLS subject.

40. A method of reducing the severity of TLS in a hematological cancer subject being treated with alvocidib, the method comprising:
A. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
B. administering to the subject an effective amount of one or more TLS therapies if the subject has an elevated serum potassium level or an abnormal laboratory TLS panel.

41. A method of monitoring for TLS while treating a hematological cancer in a subject with alvocidib, the method comprising:
A. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
B. administering to the subject an effective amount of one or more TLS therapies if the subject has an elevated serum potassium level or an abnormal laboratory TLS panel.

42. A method of treating TLS in a hematological cancer subject being treated with alvocidib, the method comprising:
A. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration;
B. determining the subject has an elevated serum potassium level or an abnormal laboratory TLS panel; and
C. administering to the subject an effective amount of one or more TLS therapies.

43. A method of decreasing mortality from TLS in hematological cancer subjects being treated with alvocidib, the method comprising:
A. monitoring each subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
B. administering to each subject an effective amount of one or more TLS therapies if the subject has an elevated serum potassium level or an abnormal laboratory TLS panel.

44. A method of increasing survival time of hematological cancer subjects being treated with alvocidib, the method comprising:
A. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
B. administering to the subject an effective amount of one or more TLS therapies if the subject has an elevated serum potassium level or an abnormal laboratory TLS panel.

45. A method of reducing the incidence of TLS in hematological cancer subjects being treated with alvocidib, the method comprising:
A. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
B. administering to the subject an effective amount of one or more TLS therapies if the subject has an elevated serum potassium level or an abnormal laboratory TLS panel.

46. A method of preventing TLS in a hematological cancer subject being treated with alvocidib, the method comprising:
A. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
B. administering to the subject an effective amount of one or more TLS therapies if the subject has an elevated serum potassium level or an abnormal laboratory TLS panel.

47. A method of diagnosing and treating TLS in a hematological cancer subject being treated with alvocidib, the method comprising:
A. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
B. administering to the subject an effective amount of one or more TLS therapies if the subject has an elevated serum potassium level or an abnormal laboratory TLS panel.

48. The method of any one of claims 1 to 47, wherein the hematological cancer is selected from myelodysplastic syndrome (MDS) and acute myeloid leukemia (AML).

49. The method of claim 48, wherein the AML is selected from primary AML, refractory AML, or relapsed AML.

50. The method of claim 49, wherein the AML is refractory AML or relapsed AML.

51. The method of any one of claims 1 to 47, wherein the hematological cancer is multiple myeloma.

52. The method of any one of claims 1 to 51, wherein the subject is 65 years old or younger.

53. A method of treating a hematological cancer in a subject, the method comprising:
A. administering an effective amount of alvocidib to the subject once a day for three days;
B. monitoring the subject for tumor lysis syndrome (TLS) by:
i. performing a serum potassium assay on the subject at the end of the alvocidib administration and about two hours after the end of the alvocidib administration on the first day;
ii. performing a laboratory TLS panel on the subject about four hours after the end of the alvocidib administration on the first day;
iii. performing an additional laboratory TLS panel on the subject about every two hours or about every four hours after the laboratory TLS panel in (ii), until twenty-four hours after the end of the alvocidib administration on the first day; and
iv. performing an additional laboratory TLS panel on the subject about every 6 hours for two days after the last laboratory TLS panel performed in (iii); and
C. administering to the subject an effective amount of one or more TLS therapies if the subject has an elevated serum potassium level or an abnormal laboratory TLS panel.

54. The method of claim 53, wherein the method further comprises administering an effective amount of at least one cytotoxic drug to the subject after the alvocidib administrations.

55. The method of claim 54, wherein the at least one cytotoxic drug is cytarabine.

56. The method of claim 54 or 55, wherein the subject is administered an effective amount of at least two cytotoxic drugs after the alvocidib administrations.

57. The method of claim 56, wherein the at least two cytotoxic drugs are cytarabine and mitoxantrone.

58. The method of any one of claims 55 to 57, wherein the cytarabine is administered 5 days after the first day of alvocidib administration.

59. The method of any one of claims 55 to 58, wherein the cytarabine is administered as a continuous intravenous (IV) infusion for about 72 hours.

60. The method of any one of claims 55 to 59, wherein the effective amount of cytarabine is 667 mg/m² per 24 hours, for a total of 2 gm/m².

61. The method of any one of claims 57 to 60, wherein the mitoxantrone is administered about 12 hours after the end of the cytarabine administration.

62. The method of any one of claims 57 to 61, wherein the mitoxantrone is administered as an IV infusion over about 1 to about 2 hours.

63. The method of any one of claims 57 to 62, wherein the effective amount of mitoxantrone is 40 mg/m².

64. The method of any one of claims 53 to 63, wherein the method further comprises performing an additional laboratory TLS panel on the subject every 12 hours after the last laboratory TLS panel in (iv) for at least two days.

65. The method of claim 64, wherein the additional laboratory TLS panel is performed every 12 hours for at least five days.

66. A method of treating a hematological cancer in a subject with high risk for developing TLS, the method comprising:
A. administering an effective amount of alvocidib to the subject;
B. monitoring the subject for tumor lysis syndrome (TLS) by:
i. performing a serum potassium assay on the subject at the end of the alvocidib administration and about two hours after the end of the alvocidib administration;
ii. performing a laboratory TLS panel on the subject about four hours after the end of the alvocidib administration;
iii. performing an additional laboratory TLS panel on the subject about every two hours after the laboratory TLS panel in (ii), until twenty-four hours after the end of the alvocidib administration; and
iv. performing an additional laboratory TLS panel on the subject about every 6 hours for two days after the last laboratory TLS panel performed in (iii); and
C. administering to the subject an effective amount of one or more TLS therapies if the subject has an elevated serum potassium level or an abnormal laboratory TLS panel.

67. A method of treating a hematological cancer in a subject without high risk for developing TLS, the method comprising:
A. administering an effective amount of alvocidib to the subject;
B. monitoring the subject for tumor lysis syndrome (TLS) by:
i. performing a serum potassium assay on the subject at the end of the alvocidib administration and about two hours after the end of the alvocidib administration;
ii. performing a laboratory TLS panel on the subject about four hours after the end of the alvocidib administration;
iii. performing an additional laboratory TLS panel on the subject about every four hours after the laboratory TLS panel in (ii), until twenty-four hours after the end of the alvocidib administration; and
iv. performing an additional laboratory TLS panel on the subject about every 6 hours for two days after the last laboratory TLS panel performed in (iii); and
C. administering to the subject an effective amount of one or more TLS therapies if the subject has an elevated serum potassium level or an abnormal laboratory TLS panel.

68. A method of treating frontline, relapsed or refractory AML, in a subject, the method comprising:
A. administering an effective amount of alvocidib to the subject;
B. monitoring the subject for tumor lysis syndrome (TLS) by:
i. performing a serum potassium assay on the subject at the end of the alvocidib administration and about two hours after the end of the alvocidib administration; and
ii. performing a laboratory TLS panel on the subject about four hours after the end of the alvocidib administration; and
C. administering to the subject an effective amount of one or more TLS therapies if the subject has an elevated serum potassium level or an abnormal laboratory TLS panel, wherein:
if the subject exhibits evidence of clinically meaningful TLS after the alvocidib administration, performing an additional laboratory TLS panel on the subject about every two hours after the laboratory TLS panel in (ii), until twenty-four hours after the end of the alvocidib administration, and
if the subject does not exhibit evidence of clinically meaningful TLS after the alvocidib administration, performing an additional laboratory TLS panel on the subject about every four hours after the laboratory TLS panel in (ii), until twenty-four hours after the end of the alvocidib administration.

69. A method of treating a hematological cancer in a subject comprising:
A. administering an effective amount of alvocidib to the subject;
B. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
C. administering to the subject an effective amount of one or more TLS therapies if the subject has an abnormal laboratory TLS panel.

70. The method of claim 69, wherein the monitoring further comprises performing an additional laboratory TLS panel on the subject weekly after the first week following the alvocidib administration.

71. The method of claim 69 or 70, wherein the monitoring further comprises performing a laboratory TLS panel on the subject prior to the alvocidib administration.

72. The method of any one of claims 69 to 71, wherein the subject exhibits evidence of clinically meaningful TLS after the alvocidib administration.

73. The method of any one of claims 69 to 72, wherein the laboratory TLS panel includes a potassium assay, further comprising administering to the subject an effective amount of one or more TLS therapies if the subject has an elevated serum potassium level.

74. The method of claim 73, wherein the elevated serum potassium level is selected from greater than 4.0 mEq/L, greater than 5.0 mEq/L, greater than 5.5 mEq/L, or a level that is at least 25% above the subject's baseline level.

75. The method of claim 74, wherein the elevated serum potassium level is greater than 4.0 mEq/L, and the one or more TLS therapies comprise administering to the subject about 30 g of sodium polystyrene sulfonate.

76. The method of claim 75, wherein the elevated serum potassium level is greater than 5.0 mEq/L, and the one or more TLS therapies further comprise administering to the subject about 10 units of intravenous (IV) rapid-acting insulin and about 25 g of IV dextrose 50%.

77. The method of claim 76, wherein the elevated serum potassium level is greater than 5.5 mEq/L, and the one or more TLS therapies further comprise emergent, intermittent or continuous dialysis.

78. The method of any one of claims 69 to 77, wherein the method further comprises performing a lactic acid dehydrogenase (LDH) assay on the subject at least once every 24 hours after the end of alvocidib administration.

79. The method of any one of claims 69 to 78, wherein the method further comprises administering to the subject IV hydration.

80. The method of claim 79, wherein the IV hydration administration begins about two hours prior to the start of the alvocidib administration, and continues for from about one hour to about two hours.

81. The method of claim 79 or 80, wherein the IV hydration is administered for from about one hour to about two hours, beginning at the end of the alvocidib administration.

82. The method of any one of claims 79 to 81, wherein the method further comprises administering to the subject an effective amount of allopurinol beginning about 72 hours prior to the start of the alvocidib administration.

83. The method of any one of claims 79 to 82, wherein the method further comprises administering to the subject an effective amount of an oral phosphate binder, beginning at the start of the administration of IV hydration.

84. The method of any one of claims 69 to 83, wherein the method further comprises performing a fibrinogen assay on the subject prior to the start of the alvocidib administration.

85. The method of any one of claims 69 to 84, wherein the effective amount of alvocidib is administered to the subject as a 30-60-minute IV bolus of alvocidib.

86. The method of claim 85, wherein the IV bolus is from about 20 mg/m² to about 60 mg/m².

87. The method of claim 86, wherein the IV bolus is about 25 mg/m² of alvocidib.

88. The method of claim 86, wherein the IV bolus is about 50 mg/m² of alvocidib.

89. The method of any one of claims 69 to 88, wherein the effective amount of alvocidib is administered to the subject once weekly.

90. The method of any one of claims 69 to 89, wherein the effective amount of alvocidib is administered to the subject as a 30-60-minute IV bolus comprising about 25 mg/m² alvocidib on day 1 of a treatment cycle; and as a 30-60-minute IV bolus comprising about 50 mg/m² alvocidib on days 8 and 15 of the treatment cycle.

91. The method of any one of claims 69 to 89, wherein the effective amount of alvocidib is administered to the subject as a 30-60-minute IV bolus comprising about 25 mg/m² alvocidib on day 1 of a treatment cycle; and as a 30-60-minute IV bolus comprising about 50 mg/m² alvocidib on day 15 of the treatment cycle.

92. The method of any one of claims 69 to 91, wherein the method further comprises administering to the subject an effective amount of at least one cytotoxic drug after the alvocidib administration.

93. The method of claim 92, wherein the at least one cytotoxic drug is cytarabine.

94. The method of claim 93, wherein the cytarabine is administered once daily for ten days.

95. The method of claim 93 or 94, wherein the cytarabine is administered as a subcutaneous injection.

96. The method of any one of claims 93 to 95, wherein about 20 mg/m² cytarabine is administered to the subject per day.

97. The method of any one of claims 93 to 96, wherein the effective amount of cytarabine is about 20 mg/m² per day once daily for ten days, for a total of 200 mg/m².

98. The method of any one of claims 69 to 97, wherein the method further comprises obtaining a biological sample from the subject prior to the alvocidib administration, and determining a MCL-1 dependence score from the sample.

99. The method of claim 98, wherein the MCL-1 dependence score is selected from greater than or equal to 40%, 30% to less than 40%, 15% to less than 30%, and 0 to less than 15%.

100. The method of any one of claims 69 to 99, wherein the method further comprises determining whether the subject is a high risk TLS subject prior to the alvocidib administration.

101. The method of any one of claims 69 to 100, wherein the subject is a high risk TLS subject.

102. A method of reducing the severity of TLS in a hematological cancer subject being treated with alvocidib, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. administering to the subject an effective amount of one or more TLS therapies if the subject has an abnormal laboratory TLS panel.

103. A method of monitoring for TLS while treating a hematological cancer in a subject, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. administering to the subject an effective amount of one or more TLS therapies if the subject has an abnormal laboratory TLS panel.

104. A method of treating TLS in a hematological cancer subject being treated with alvocidib, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. determining the subject has an abnormal laboratory TLS panel; and
C. administering to the subject an effective amount of one or more TLS therapies.

105. A method of decreasing mortality from TLS in hematological cancer subjects being treated with alvocidib, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. administering to each subject an effective amount of one or more TLS therapies if the subject has an abnormal laboratory TLS panel.

106. A method of increasing survival time of hematological cancer subjects being treated with alvocidib, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. administering to the subject an effective amount of one or more TLS therapies if the subject has an abnormal laboratory TLS panel.

107. A method of reducing the incidence of TLS in hematological cancer subjects being treated with alvocidib, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. administering to the subject an effective amount of one or more TLS therapies if the subject has an abnormal laboratory TLS panel.

108. A method of preventing TLS in a hematological cancer subject being treated with alvocidib, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. administering to the subject an effective amount of one or more TLS therapies if the subject has an abnormal laboratory TLS panel.

109. A method of diagnosing and treating TLS in a hematological cancer subject being treated with alvocidib, the method comprising:
A. monitoring the subject for tumor lysis syndrome (TLS) by:
i. performing a laboratory TLS panel on the subject from three to about four hours after the end of the alvocidib administration; and
ii. performing an additional laboratory TLS panel on the subject daily for three days following the alvocidib administration; and
B. administering to the subject an effective amount of one or more TLS therapies if the subject has an abnormal laboratory TLS panel.

110. The method of any one of claims 69 to 109, wherein the hematological cancer is selected from myelodysplastic syndrome (MDS) and acute myeloid leukemia (AML).

111. The method of claim 110, wherein the AML is selected from primary AML, refractory AML, or relapsed AML.

112. The method of claim 111, wherein the AML is refractory AML or relapsed AML.

113. The method of any one of claims 69 to 109, wherein the hematological cancer is multiple myeloma.

114. The method of any one of claims 69 to 113, wherein the subject is older than 65-years old.

115. The method of any one of claims 40 to 52 and 102 to 114, further comprising administering an effective amount of alvocidib to the subject.

116. The method of any one of claims 53 to 85, 92 to 101, and 115, wherein the effective amount of alvocidib is administered as one or more hybrid doses.

117. A method of treating a hematological cancer in a subject comprising:
A. administering a prophylactically effective amount of each of: intravenous (IV) hydration, allopurinol and an oral phosphate binder to the subject;
B. administering a therapeutically effective amount of alvocidib to the subject;
D. monitoring the subject for tumor lysis syndrome (TLS) by performing a laboratory TLS panel on the subject about three to about four hours after the end of the alvocidib administration; and
E. administering to the subject a therapeutically effective amount of one or more TLS therapies if the subject has an abnormal laboratory TLS panel.

118. The method of any one of claims 1 to 117, wherein the subject is a human.
